(19) United States
(12) Patent Application Publication
(10) Pub. No.: US 2020/0246268 A1
(43) Pub. Date: Aug. 6, 2020

(54) CPZEN COMPOSITIONS AND USES

(71) Applicants: PAI Life Sciences Inc., Seattle, WA (US); Infectious Disease Research Institute, Seattle, WA (US); Research Triangle Institute, Research Triangle Park, NC (US)

(72) Inventors: Darrick CARTER, Seattle, WA (US); Anthony James HICKEY, Chapel Hill, NC (US); Phillip Gregory DURHAM, Sanford, NC (US); Ragan PITNER, Seattle, WA (US)

(21) Appl. No.: 16/620,160

(22) PCT Filed: Jun. 7, 2018
(86) PCT No.: PCT/US2018/036451

§ 371 Date: Dec. 6, 2019

Related U.S. Application Data
(60) Provisional application No. 62/517,136, filed on Jun. 8, 2017.

(51) Int. Cl. A61K 9/14; A61K 31/7076; A61K 38/12; A61K 9/00

(52) U.S. Cl. CPC A61K 9/14; A61K 31/7076; A61K 45/06; A61K 9/0073; A61K 38/12

(57) ABSTRACT

Capreomycin sulfate and CPZEN-45, which act in non-identical manners to treat tuberculosis infection, are combined into particles by spray drying thereby giving an intimate mixture for combination drug therapy. The spray dried combination powder is prepared in an aerodynamic particle size range (such as 1-5 μm) suitable for pulmonary delivery when delivered from an inhaler.

### STATEMENT OF GOVERNMENT RIGHTS

This invention was made with U.S. Government support from the National Institute of Allergy and Infectious Diseases National Institutes of Health, Department of Health and Human Services, under Contract No.'s HHSN272201300014C and HHSN272201500030C to DC and AH. The U.S. Government has certain rights in this invention.

### FIELD OF THE INVENTION

The present invention relates generally to pharmaceutical compositions and methods for treating and preventing disease such as tuberculosis.

### BACKGROUND

Tuberculosis (TB) is currently the single most serious infectious disease attributable to a single-causative organism. One third of the world's population is infected with tuberculosis (TB) (WHO, 2016). 7-8 million people manifest symptoms of disease and 2 million die annually (Misra et al., 2011, WHO, 2016).

Treating and preventing TB is complicated by the fact that the causative agent has become increasingly resistant to the activity of drugs. While therapy for TB is effective in drug susceptible disease, the incidence of drug resistant and extensively drug resistant TB is increasing (Muttil et al., 2009) and current therapy is of long duration often leading to poor patient compliance. Moreover, the treatment of patients who are co-infected with both HIV and TB is a particularly urgent unmet need. Therefore, there is a critical need for new approaches to treat various forms of the disease.

First line therapy for TB may involve a combination of multiple drugs. A common combination is isoniazid and rifampicin in combination with pyrazinamide and ethambutol (Hickey and Smyth, 2010, 2010). Monotherapy in most cases cannot be successfully employed to treat the disease and leads to a high probability of recurrence and induced drug resistance. In drug resistant disease a second line of treatment is required in which other drugs are added to the regimen, such as capreomycin, ethionamide, and streptomycin (Hickey and Smyth, 2010). Therapy lasts six months and requires direct observation and oversight to be effective. These long, inconvenient, and often uncomfortable treatments have a negative impact on patient compliance and consequently encourage the generation of drug-resistant TB (Boehme et al., 2011, Lawn et al., 2013).

The invention of individual new drugs is the prevailing strategy in the fight against the increasing incidence of multiple drug-resistant (MDR) and extensively drug-resistant (XDR) TB. Several candidates are in development but it is not clear whether, and when, they will receive the regulatory approval required for their use in new drug regimens. Further, since monotherapy is to be avoided as rapidly inducing drug resistance which could be counterproductive for future therapeutic efforts.

There thus remains a need in the art for more effective treatments for TB, particularly drug-resistant TB and extensively drug-resistant TB. The present disclosure addresses this need. In addition to tuberculosis, there is also a need for further compositions to treat other bacterial infections, such as Mycobacteria infections, including non-tuberculosis Mycobacteria (NTM) infections. The present disclosure encompasses new compositions comprising combinations of active pharmaceutical ingredients (APIs) that may be effective in treating bacterial infections, such as bacterical infections affecting the lungs, Mycobacteria infections, NTMs, tuberculosis, MDR tuberculosis and XDR tuberculosis.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

In brief, the present disclosure provides compositions, methods for preparing the compositions, and methods for treating disease. The compositions include a CPZEN such as CPZEN-45, and at least one other active pharmaceutical ingredient (API), such as at least one antibiotic. The method for preparing the composition may comprise spray drying a solution containing both CPZEN and API to provide a solid composition. The disease may be, for example a bacterial infection, such as a non-tuberculosis Mycobacteria infection, or tuberculosis, such as multidrug resistant (MDR) tuberculosis, or extensively drug resistant (XDR) tuberculosis. The compositions may be administered, for example, by inhalation, nebulization, by parenteral administration, topical administration, or injection, and may be administered as an aerosolized droplet, or as a powder via inhalation.

In one embodiment, the present disclosure provides a composition, e.g., a pharmaceutical composition, which comprises a CPZEN, e.g., CPZEN-45, and a second active pharmaceutical ingredient, e.g., at least one other antibiotic. One or more of the following optional features may be used to describe the composition of the present disclosure: the composition contains three active pharmaceutical agents, e.g., two other antibiotics; CPZEN comprises 10-90 wt % of a total weight of active pharmaceutical ingredient in the composition; CPZEN is in a free base form; CPZEN is in a hydrochloride salt form, where in each case the CPZEN may be CPZEN-45. In addition, the at least one other antibiotic is an aminoglycoside; the antibiotic is an aminoglycoside is selected from one or more of capreomycin, isoniazid, pyrazinamide, clarithromycin, azithromycin, rifampin, rifabutin, ethambutol, levofloxacin, moxifloxacin, ofloxacin, clofazimine, clarithromycin, cycloserine, para-aminosalicylic acid, terizidone, thionamide, protionamide, gatifloxacin, bedaquiline, delamanid, meropenem, kanamycin , amikacin, tobramycin, dibekacin, gentamicin, sisomicin, netilmicin, neomycins B, C, neomycin E (paromomycin) and streptomycin; the antibiotic has anti-mycobacterial activity. In one embodiment, the antibiotic is a capreomycin, e.g., a capreomycin salt such as capreomycin sulfate. The composition may additionally, or alternatively, be characterized in terms of its activity, e.g., the composition is effective against tuberculosis; the composition is effective against tuberculosis which is multiple drug-resistant (MDR) tuberculosis; the composition is effective against tuberculosis which is extensively or extremely drug-resistant (XDR) tuberculosis; the composition is effective against non-tuberculosis mycobacterial infection. Furthermore, the composition may additionally or alternatively be characterized by one or more of the following: the composition consists essentially of CPZEN, e.g., CPZEN-45, and one other antibiotic effective against tuberculosis; the composition also includes an excipient, where the excipient is present in a minor amount, e.g., less than 10 wt % of the composition; the composition does not include any excipient. The composition may also be characterized in terms of its form, e.g., the composition is in the form of a powder; the composition is a powder that contains less than 10 wt % water based on the total weight of the composition; the composition is a spray-dried powder; the composition is a spray-dried powder having an average particle size between 0.5 and 10 p.m; the composition is a pharmaceutical composition in a unit dosage form, wherein the dose of CPZEN, e.g., CPZEN-45, is 0.1-10 g in the unit dosage form; the composition may be stored in a container, where the container does not allow moisture from the environment to contact the composition. These and other exemplary features of the composition are described in additional detail herein.

In another embodiment, the present disclosure provides a method of treating bacterial infections, such as tuberculosis or non-tuberculosis mycobacterial infections. The tuberculosis may be selected from “regular” tuberculosis, multiple drug resistant (MDR) tuberculosis, extensively drug resistant (XDR) tuberculosis. In another embodiment, the present disclosure provides a method of treating Mycobacteria infections. For example, the present disclosure provides a method of treating a bacterial infection comprising administering to a subject in need thereof a composition as described herein, comprising CPZEN such as CPZEN-45 and at least one other antibiotic as described above. Optionally, the therapy may be administered to the subject via a route selected from inhalation, nebulization, parenteral administration, topical administration, and injection. Optionally, the therapy may be administered to the subject in a form of an aerosolized droplet. Optionally, the therapy may be administered to the subject as a powder via inhalation. Regardless of how the therapy is administered, the composition to be administered may or may not further comprise excipients.

Exemplary embodiments of this disclosure further include the following:

1. A composition comprising CPZEN-45 and at least one other antibiotic. 2. The composition of embodiment 1, wherein CPZEN-45 comprises 10-90 wt % of a total weight of active pharmaceutical ingredient in the composition. 3. The composition of embodiment 1 or 2, wherein CPZEN-45 is in a free base form. 4. The composition of embodiment 1 or 2, wherein CPZEN-45 is in a hydrochloride salt form. 5. The composition of any one of embodiments 1-4, wherein the at least one other antibiotic comprises an aminoglycoside. 6. The composition of embodiment 5, wherein the aminoglycoside is selected from capreomycin, isoniazid, pyrazinamide, clarithromycin, azithromycin, rifampin, rifabutin, ethambutol, levofloxacin, moxifloxacin, ofloxacin, clofazimine, clarithromycin, cycloserine, para-aminosalicylic acid, terizidone, thionamide, protionamide, gatifloxacin, bedaquiline, delamanid, meropenem, kanamycin , amikacin, tobramycin, dibekacin, gentamicin, sisomicin, netilmicin, neomycins B, C, neomycin E (paromomycin) and streptomycin. 7. The composition of any one of embodiments 1-6, wherein the at least one other antibiotic comprises capreomycin sulfate. 8. The composition of any one of embodiments 1-4, wherein the at least one other antibiotic consists essentially of capreomycin sulfate. 9. The composition of any one of embodiments 1-8, wherein the at least one other antibiotic has anti-mycobacterial activity. 10. The composition of any one of embodiments 1-9, which is effective against tuberculosis. 11. The composition of embodiment 10, which is effective against multiple drug-resistant (MDR) tuberculosis. 12. The composition of embodiment 10, which is effective against extensively drug-resistant (XDR) tuberculosis. 13. The composition of any one of embodiments 1-12, which is effective against non-tuberculosis mycobacterial disease. 14. The composition of any one of embodiments 1-13, consisting essentially of CPZEN-45 and one other antibiotic effective against tuberculosis. 15. The composition of any one of embodiments 1-13, further comprising an excipient, the excipient present in less than 10 wt % of the composition. 16. The composition of any one of embodiments 1-13, consisting essentially of CPZEN-45 and one other antibiotic effective against tuberculosis and at least one excipient, wherein the excipient is optionally present in less than 10 wt % of the composition. 17. The composition of any one of embodiments 1-14, which does not include an excipient. 18. The composition of any one of embodiments 1-17 in a form of a powder. 19. The composition of embodiment 18, wherein the powder comprises particles, the particles comprising both CPZEN-45 and one other antibiotic, wherein the one other antibiotic is optionally capreomycin sulfate. 20. The composition of any one of embodiments 1-19, which contains less than 10 wt % water. 21. The composition of any one of embodiments 1-20, which is a spray-dried powder. 22. The composition of embodiment 21, wherein the spray-dried powder has an average particle size between 0.5 and 10 μm. 23. A composition of any of embodiments 1-22 in a unit dosage form, wherein the dose of CPZEN-45 is between 0.1 g to 10 g. 24. A composition of any of embodiments 1-23 in a container, wherein the composition is stored in the container in a manner that excludes water entering the container. 25. A composition of any of embodiments 1-24 for use in treating a bacterial infection. 26. The composition for use of embodiment 25, wherein the bacterial infection is a Mycobacteria infection. 27. The composition for use of embodiment 25 or 26, wherein the treatment comprises administering the composition to the subject via a route selected from inhalation, nebulization, parenteral administration, topical administration, and injection. 28. The composition for use of embodiment 25 or 26, wherein the treatment comprises administering the composition in a form of an aerosolized droplet. 29. The composition for use of embodiment 25 or 26, wherein the treatment comprises administering the composition as a powder via inhalation. 30. A method of treating a bacterial infection comprising administering to a subject in need thereof a composition comprising CPZEN-45 and at least one other antibiotic according to any of embodiments 1-24. 31. The method of embodiment 30, wherein the bacterial infection is a Mycobacteria infection. 32. The method of embodiment 30 or 31, wherein the composition is administered to the subject via a route selected from inhalation, nebulization, parenteral administration, topical administration, and injection. 33. The method of embodiment 30 or 31, wherein the composition is administered to the subject in a form of an aerosolized droplet. 34. The method of embodiment 30 or 31, wherein the composition is administered to the subject as a powder via inhalation.

Additional exemplary embodiments include the following: A spray-dried powder composition comprising CPZEN-45 and capreomycin. In some embodiments, the CPZEN-45 comprises 10-90 wt %, 20-80 wt %, 30-70 wt %, 40-60 wt %, or 45-55 wt % of total weight of active pharmaceutical ingredient in the composition. In some embodiments, the CPZEN-45 comprises 40-60 wt %, 45-55 wt %, 45 wt %, 46 wt %, 47 wt %, 48 wt %, 49 wt %, 50 wt %, 51 wt %, 52 wt %, 53 wt %, 54 wt %, or 55 wt % of total weight of active pharmaceutical ingredient in the composition, or wherein the CPZEN-45 and capreomycin are present in a weight ratio range of 40:60 to 60:40, or a weight ratio range of 45:55 to 55:45, or a weight ratio range of 48:52 to 52:48, or a weight ratio of 50:50. In some embodiments, the CPZEN-45 and capreomycin make up 100% of the total weight of active pharmaceutical ingredient of the composition. In some embodiments, the composition consists essentially of CPZEN-45 and capreomycin. In some embodiments, the CPZEN-45 is in a free base form. In others, it is in a salt form, such as a hydrochloride salt form. In some embodiments, capreomycin is in a salt form, such as a sulfate salt form (capreomycin sulfate). In some embodiments, the composition further comprises at least one other antibiotic. In some embodiments, the at least one other antibiotic is an aminoglycoside. In some embodiments, the aminoglycoside is selected from isoniazid, pyrazinamide, clarithromycin, azithromycin, rifampin, rifabutin, ethambutol, levofloxacin, moxifloxacin, ofloxacin, clofazimine, clarithromycin, cycloserine, para-aminosalicylic acid, terizidone, thionamide, protionamide, gatifloxacin, bedaquiline, delamanid, meropenem, kanamycin, amikacin, tobramycin, dibekacin, gentamicin, sisomicin, netilmicin, neomycins B, C, neomycin E (paromomycin) and streptomycin. In some embodiments, the composition comprises less than 5 wt % excipients, which comprises less than 4 wt % excipients, which comprises less than 3 wt % excipients, which comprises less than 2 wt % excipients, or which comprises less than 1 wt % excipients, based on the total weight of the composition; or which does not comprise excipients. In some embodiments, the composition does not comprise an amino acid excipient, does not comprise a surfactant excipient, or does not comprise either an amino acid or a surfactant excipient. In some embodiments, the composition contains less than 10 wt % waterbased on the total weight of the composition. In some embodiments, the spray-dried powder composition comprises particles with an average particle size between 0.1 and 10 μm, between 0.5 and 10 μm, between 1 and 5 μm, or between 2 and 4 μm. In some embodiments, the spray-dried composition consists essentially of CPZEN-45 salt (e.g. HCl salt) and capreomycin salt (e.g. sulfate salt) and less than 1, 2, 3, 4, or 5 wt % water, wherein the CPZEN-45 and capreomycin are present at a weight ratio range of 45:55 to 55:45, or a weight ratio range of 48:52 to 52:48, or a weight ratio range of 50:50, and wherein the powder has a particle size of between 1 and 5 μm. In some embodiments, the spray-dried powder composition is at least 95%, at least 96%, or at least 97% stable against degradation of CPZEN-45 and capreomycin for at least 6 months at 25° C. and 60% relative humidity. In some embodiments, the CPZEN-45 and capreomycin are mixed without addition of excipient prior to forming the spray-dried powder. In some embodiments, the spray-dried powder is formed from a mixture consisting essentially of CPZEN-45 and capreomycin in water. This disclosure also includes a pharmaceutical composition comprising a unit dosage form of the composition as described above. In some embodiments, such as pharmaceutical composition comprises a unit dose of CPZEN-45 of 0.1-10 g, 1-10 g, or 1-5 g, and/or comprising a unit dose of capreomycin of 0.1-10 g, 1-10 g, or 1-5 g. This disclosure also contemplates a container comprising the pharmaceutical composition, for example, containing a unit dose of the composition.

In some embodiments, the composition or pharmaceutical composition or container is intended for use in treating a bacterial infection. In some embodiments, the bacterial infection is a Mycobacteria infection. In some embodiments, the Mycobacteria infection is a non-tuberculosis Mycobacteria (NTM) infection, for example, one or more of M. abscessus, M. abscessus massiliense, M. chelonae, M. kansasii, M. xenopii, M. intracellulare, M. fortuitum, M. ulcerans, M. smegmatis, M. marinum, M. peregrinum, M. mucogenicum, M. alvei, M. porcinum, M. septicum, M. wolinskyi, M. lentiflavum, M. mageritense, M. phlei, M. vaccae, M. malmoense, M. gordonae, M. simiae, M. scrofulaceum, M. hibermiae, M. bovis, and M. avium. In some embodiments, the bacterial infection is tuberculosis, MDR tuberculosis, or XDR tuberculosis. In some embodiments, treating the bacterial infection comprises administering the composition or pharmaceutical composition to the subject via a route selected from inhalation, nebulization, parenteral administration, topical administration, and injection. In some embodiments, treating the bacterial infection comprises administering the composition or pharmaceutical composition in a form of an aerosolized droplet. In some embodiments, treating the bacterial infection comprises administering the composition as a powder via inhalation.

The disclosure also contemplates methods of treating a bacterial infection comprising administering to a subject in need thereof the composition or pharmaceutical composition as described above. In some embodiments, the bacterial infection is a Mycobacteria infection. In some embodiments, the Mycobacteria infection is a non-tuberculosis Mycobacteria (NTM) infection, for example, one or more of M. abscessus, M. abscessus massiliense, M. chelonae, M. kansasii, M. xenopii, M. intracellulare, M. fortuitum, M. ulcerans, M. smegmatis, M. marinum, M. peregrinum, M. mucogenicum, M. alvei, M. porcinum, M. septicum, M. wolinskyi, M. lentiflavum, M. mageritense, M. phlei, M. vaccae, M. malmoense, M. gordonae, M. simiae, M. scrofulaceum, M. hibermiae, M. bovis, and M. avium. In some embodiments, the bacterial infection is tuberculosis, MDR tuberculosis, or XDR tuberculosis. In some embodiments, the composition is administered to the subject via a route selected from inhalation, nebulization, parenteral administration, topical administration, and injection. In some embodiments, the composition is administered to the subject in a form of an aerosolized droplet. In some embodiments, the composition is administered to the subject as a powder via inhalation.

The present disclosure also includes a method of preparing a spray-dried composition of CPZEN-45 and capreomycin comprising obtaining a CPZEN-45 salt and a capreomycin salt in a weight ratio range of 30:70 to 70:30, or of 40:60 to 60:40, or of 45:55 to 55:45, or of 48:52 to 52:48, or a weight ratio of 50:50, preparing a feed solution comprising the CPZEN-45 salt and the capreomycin salt at the above weight ratio in water or buffered aqueous solution, wherein the feed solution optionally does not comprise an excipient or does not comprise an amino acid excipient and/or a surfactant excipient and optionally consists essentially of the CPZEN-45 salt and the capreomycin salt in the water or the buffered aqueous solution, subjecting the feed solution to spray drying, and collecting resulting spray-dried particles. In some embodiments, this method can be used to prepare the compositions and pharmaceutical compositions described above.

Further information regarding these compositions and methods is provided in the Detailed Description that follows. All references disclosed herein are hereby incorporated by reference in their entirety as if each was incorporated individually.

This Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

Additional details of certain embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments.

### BRIEF DESCRIPTION OF THE DRAWINGS

FIG. 1 shows a plot of weight change in four different groups of six guinea pigs given either no treatment (n.t.) or given 20 mg/kg of each of CPZEN-45 and capreomycin via intramuscular injection (20/20 i.m.), 2 mg/kg of each of CPZEN-45 and capreomycin via intramuscular injection (2/2 i.m.), or inhale capreomycin/CPZEN-45. The CPZEN-45/capreomycin is a spray-dried powder composition comprising 50:50 by weight mixture of the two active agents.

FIGS. 2A and 2B show bacterial burden in the tested guinea pigs treated as described for FIG. 1 above, measured as CFU in the right cranial lobe (FIG. 2A) or spleen (FIG. 2B).

### DETAILED DESCRIPTION OF CERTAIN EMBODIMENTS

The present disclosure provides, for example, compositions, methods for preparing the compositions, and methods for treating bacterial infections.

### Definitions

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

All references disclosed herein, including patent references and non-patent references, are hereby incorporated by reference in their entirety as if each was incorporated individually.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

In this disclosure, the terms below have the following definitions.

The term “CPZEN-45” refers to a particular compound disclosed, for example, in U.S. Pat. No. 9,040,502. CPZEN-45 is also known as Caprazene-45. CPZEN-45 is a type of CPZEN (caprazene). The term refers to all forms of CPZEN-45 including its free base and salt forms and ionized salt forms when the compound is in solution. Similarly, the term “CPZEN” refers to all forms of a CPZEN compound. The term “capreomycin” likewise refers to all forms of the compound including free base and salt forms and ionized salt forms when the compound is in solution.

An “active pharmaceutical ingredient” or “active pharmaceutical agent” or “active agent” or “API” refers to a compound or mixture of compounds, such as CPZEN-45 or capreomycin or a combination of the two, that is intended to have a treatment effect in a subject with a disease or disorder. For example, these terms may be used to refer to the portion of a composition that is made up of compound(s) intended to have such a treatment effect.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided, however.

“Treatment,” as used herein, refers to therapeutic treatment, for example, wherein the object is to reduce in severity or slow progression of the targeted pathologic condition or disorder or improve at least one symptom of the disorder as well as, for example, wherein the object is to prevent or inhibit onset of a condition or disorder for example after exposure or possible exposure to a causative agent. In certain embodiments, the term “treatment” covers any administration or application of a therapeutic for disease in a patient, and includes inhibiting or slowing the disease or progression of the disease; partially or fully relieving the disease, improving at least one symptom of the disease, or causing the disease to plateau to have reduced severity. The term “treatment” also includes reducing the severity of any phenotypic characteristic and/or reducing the incidence, degree, or likelihood of that characteristic. Those in need of treatment include those already with the disorder as well as those at risk of onset of the disorder and those in whom a recurrence of the disorder is to be prevented or slowed down.

The term “effective amount” or “therapeutically effective amount” refers to an amount of an active pharmaceutical agent effective to treat a disease or disorder in a subject.

As used herein, a “multi-drug-resistant (MDR) tuberculosis” means a TB caused by a bacterial strain that is resistant to treatment with at least isoniazid and rifampin, as noted in the current TB fact sheet of the Centers for Disease Control (CDC). An “extensively-drug-resistant (XDR) tuberculosis” means a TB caused by a strain that is resistant to isoniazid and rifampin as well as fluoroquinolone and at least one injectable second-line drug such as amikacin, kanamycin, and capreomycin, as noted in the current CDC TB fact sheet, available at www (dot) cdc (dot) gov (slash) tb (slash) publications (slash) factsheets (slash) drtb (slash) mdrtb (dot) htm (accessed Jun. 2, 2018).

An “excipient” in the context of the spray-dried powder compositions of this disclosure refers to a non-toxic solid, semisolid, or liquid filler, diluent, encapsulating material, formulation auxiliary, stabilizing agent, or carrier conventional in the art for preparation of a composition intended for pharmaceutical use. An excipient, for example, may comprise a surfactant, i.e. a molecule such as an amphiphilic polymer to aid in the solubility of an active pharmaceutical agent, e.g. PEG, Tween®, a phospholipid, and the like. An excipient may also comprise agents such as amino acids, sugars, sugar alcohols and the like that may also help to solubilize or provide stability to a composition. In some embodiments herein, a spray-dried powder composition does not comprise an excipient or comprises only a small amount of excipient by weight compared to the active pharmaceutical agents.

Additional terms are defined below.

### Compositions

Compositions herein may include a CPZEN such as CPZEN-45, and another active pharmaceutical ingredient (API). In some embodiments, CPZEN and the other active pharmaceutical ingredient treat bacterial infections such as tuberculosis via different mechanisms of action, i.e., in a non-identical manner. The method for preparing the composition may be spray drying a solution containing both CPZEN and API to provide a solid composition. For example, the composition may comprise CPZEN-45 and capreomycin, which is useful as a combination therapy. The disease may be tuberculosis, e.g., drug-resistant tuberculosis. The method of treatment may be inhalation therapy. For example, in one embodiment, capreomycin sulfate and CPZEN-45, which act in non-identical manners to treat tuberculosis infection, are combined into particles by spray drying thereby giving an intimate mixture for combination drug therapy. The spray dried combination powder is prepared in an aerodynamic particle size range (1-5 μm) suitable for pulmonary delivery when delivered from an inhaler.

Capreomycin, a cyclic peptide antibiotic thought to target the ribosome (Johansen et al., 2006), is soluble with a minimum inhibitory concentration (MIC) against TB of 2 μg/mL. Capreomycin is not orally available and so is presently delivered by intramuscular or intravenous injection. While this has proven effective, it is not only painful and inconvenient for the patient but requires cold chain storage as well as needles, syringes and other waste material which is particularly hazardous when there is a question of HIV coinfection (Fiegel et al., 2008).

CPZEN-45, a caprazamycin derivative thought to target cell wall biogenesis (Ishizaki et al., 2013) with activity against multiple forms of TB is also not orally available due to poor absorption from the gastrointestinal tract (Hanif et al., 2014). CPZEN-45 is also known as Caprazene-45.

The present disclosure describes a combination powder of capreomycin and CPZEN-45 that may be prepared by mixing the two APIs and then spray-drying them into a powder, and that may be suitable for inhaled antibiotic therapy against TB. The present disclosure also provides a dosing method, a characterized in vitro aerosol performance, measured the long-term stability of the drug combination, a demonstration that the drug combination may be readily detectable in serum at therapeutic levels, and finds that the combination has mucin binding comparable to ciprofloxacin, which is associated with minimal interference from mucin (Huang et al., 2015).

Thus, in one embodiment the present disclosure provides a composition, e.g., a pharmaceutical composition, which comprises a CPZEN, e.g., CPZEN-45, and a second active pharmaceutical ingredient, e.g., an additional (or another) antibiotic.

Optionally, CPZEN, e.g., CPZEN-45, comprises 10-90 wt % of a total weight of active pharmaceutical ingredient in the composition. In other optional embodiments, CPZEN, e.g., CPZEN-45, contributes 20-80 wt %, or 30-70 wt %, or 40-60 wt % or 45-55 wt % or 45, 46, 47, 48 49 50. 51, 52, 53, 54, or 55 wt % of the total weight of the active pharmaceutical ingredients in the composition. In one embodiment, the weight of CPZEN, e.g., CPZEN-45, and the weight of a second active pharmaceutical ingredient, e.g., an antibiotic, are about equal, i.e., within 10% or 5% of the average of their weights in the composition.

Optionally, the CPZEN, e.g., CPZEN-45, is in a free base form. In other words, it is not a salt form. As another option, the CPZEN, e.g., CPZEN-45, is in a salt form, for example a hydrochloride salt form.

Optionally, the second active pharmaceutical agent in the composition may be an antibiotic. A CPZEN is an antibiotic, so when the second active pharmaceutical agent is also an antibiotic, the second agent may be referred to as an additional antibiotic. Optionally, the second active pharmaceutical agent in the composition may be an aminoglycoside antibiotic, e.g., the antibiotic is an aminoglycoside is selected from one or more of capreomycin, isoniazid, pyrazinamide, clarithromycin, azithromycin, rifampin, rifabutin, ethambutol, levofloxacin, moxifloxacin, ofloxacin, clofazimine, clarithromycin, cycloserine, para-aminosalicylic acid, terizidone, thionamide, protionamide, gatifloxacin, bedaquiline, delamanid, meropenem, kanamycin , amikacin, tobramycin, dibekacin, gentamicin, sisomicin, netilmicin, neomycins B, C, neomycin E (paromomycin) and streptomycin; the antibiotic has anti-mycobacterial activity. Optionally, the composition contains three active pharmaceutical ingredients, for example, CPZEN-45 and two additional antibiotics.

Optionally, the composition may additionally, or alternatively, be characterized in terms of its activity. For example, in one embodiment the composition is effective against tuberculosis. The composition may be specifically effective against tuberculosis which is multiple drug-resistant (MDR) tuberculosis. Optionally, the composition is effective against tuberculosis which is extensively or extremely drug-resistant (XDR) tuberculosis. Optionally, the composition is effective against non-tuberculosis mycobacterial disease.

Optionally, the composition may additionally or alternatively be characterized by one or more of the following. The composition consists essentially of CPZEN, e.g., CPZEN-45, and one other antibiotic effective against tuberculosis. In other words, the composition contains only two active pharmaceutical ingredients, one of them being CPZEN, particularly CPZEN-45, and the other is a different antibiotic that is effective against tuberculosis. The composition also includes an excipient, where the excipient is present in a minor amount, e.g., less than 10 wt % of the composition, or less than 5 wt % of the composition. In one embodiment, the composition of the present disclosure does not contain or include any excipient.

Optionally, the composition may also be characterized in terms of its form. For example, the composition may be in the form of a powder, such as a spray-dried powder. Optionally, the powder composition contains little or no water, e.g., less than 10 wt % or less than 5 wt % of the composition is water. Optionally, the composition is a spray-dried powder. A “spray-dried powder” is a powder that has been made by a spray-drying process, for example, as disclosed herein. A spray-dried power may be prepared having a minimal amount of water, e.g., less than 10 wt %, or less than 8 wt %, or less than 6 wt %, or less than 5 wt %, or less than 4 wt %, or less than 3 wt %, or less than 2 wt %, or less than 1 wt % water based on the total weight of the composition. The powder, e.g., the spray-dried powder, may be characterized in terms of its average particle size, which is embodiments is 0.1-10 μm, 0.5-10 μm, 1-5 μm, or 2-4 μm.

Thus, some embodiments herein provide a spray-dried powder composition comprising CPZEN-45 and capreomycin. In some such embodiments, the CPZEN-45 comprises 10-90 wt %, 20-80 wt %, 30-70 wt %, 40-60 wt %, or 45-55 wt % of total weight of active pharmaceutical ingredient in the composition, such as 45 wt %, 46 wt %, 47 wt %, 48 wt %, 49 wt %, 50 wt %, 51 wt %, 52 wt %, 53 wt %, 54 wt %, or 55 wt% of total weight of active pharmaceutical ingredient in the composition, or the CPZEN-45 and capreomycin are present in a weight ratio range of 40:60 to 60:40, or a weight ratio range of 45:55 to 55:45, or a weight ratio range of 48:52 to 52:48, or a weight ratio of 50:50. In some embodiments, the CPZEN-45 and capreomycin make up 100% of the total weight of active pharmaceutical ingredient of the composition, i.e. no other API is present in the composition. In some embodiments, the composition consists essentially of CPZEN-45 and capreomycin, meaning that any other API present is only present in small enough amounts so as not to change the basic and material characteristics of the composition. In other embodiments, the composition may comprise at least one other antibiotic, such as an aminoglycoside antibiotic, such as isoniazid, pyrazinamide, clarithromycin, azithromycin, rifampin, rifabutin, ethambutol, levofloxacin, moxifloxacin, ofloxacin, clofazimine, clarithromycin, cycloserine, para-aminosalicylic acid, terizidone, thionamide, protionamide, gatifloxacin, bedaquiline, delamanid, meropenem, kanamycin, amikacin, tobramycin, dibekacin, gentamicin, sisomicin, netilmicin, neomycins B, C, neomycin E (paromomycin) and streptomycin. In such embodiments, the CPZEN-45 may be in a free base or salt form, such as an HCl salt form. The capreomycin may be in a free base or salt form such as a sulfate salt form. In some embodiments, the spray-dried composition does not comprise an excipient, or alternatively, comprises less than 5 wt %, less than 4 wt % excipients, less than 3 wt% excipients, less than 2 wt % excipients, or less than 1 wt % excipients, based on the total weight of the composition. In some embodiments, the composition does not comprise an amino acid excipient, does not comprise a surfactant excipient, or does not comprise either an amino acid or a surfactant excipient. In such cases, when an amino acid or a surfactant excipient is not included, it is meant that no such ingredients are detected in the composition beyond trace levels that would not be expected to have an excipient's effects on the composition. In some cases, no amino acid, or no surfactant, or no excipient is detectable in the composition. In some embodiments, even though the composition does not comprise an excipient, it may comprise a salt or ionized salt, such as a buffer component intended to maintain pH either before or after the spray-drying process, for example. In other embodiments, even though the composition does not comprise an excipient, it may comprise water, for example in small amounts. For example, in some embodiments, a spray-dried composition may contain less than 10 wt % water, or less than 8 wt %, or less than 6 wt %, or less than 5 wt %, or less than 4 wt %, or less than 3 wt %, or less than 2 wt %, or less than 1 wt % water, based on the total weight of the composition. In some embodiments, the spray-dried powder composition comprises particles with an average particle size between 0.1 and 10 μm, between 0.5 and 10 μm, between 1 and 5 μm, or between 2 and 4 μm. In some embodiments, the composition consists essentially of CPZEN-45 salt (e.g. HCl salt) and capreomycin salt (e.g. sulfate salt) and less than 1, 2, 3, 4, or 5 wt % water, based on the total weight of the composition, wherein the powder has a particle size of between 1 and 5 μm, and wherein the CPZEN-45 and capreomycin salts are present in a weight ratio range of 45:55 to 55:45, or a weight ratio range of 48:52 to 52:48, or a weight ratio of 50:50. In some embodiments, a CPZEN-45 and capreomycin spray-dried powder composition is at least 95%, at least 96%, or at least 97% stable against degradation of CPZEN-45 and capreomycin for at least 6 months at 25° C. and 60% relative humidity. In some embodiments, a CPZEN-45 and capreomycin spray-dried powder composition is at least 95%, at least 96%, or at least 97% stable against degradation of CPZEN-45 and capreomycin for at least 6 months at40° C. and 75% relative humidity. In some embodiments, a CPZEN-45 and capreomycin spray-dried powder composition is at least 95%, at least 96%, or at least 97% stable against degradation of CPZEN-45 and capreomycin for at least 3 months at 25° C. and 60% relative humidity. In some embodiments, a CPZEN-45 and capreomycin spray-dried powder composition is at least 95%, at least 96%, or at least 97% stable against degradation of CPZEN-45 and capreomycin for at least 3 months at40° C. and 75% relative humidity.

In some embodiments, a spray-dried composition of CPZEN-45 and capreomycin is prepared by a process comprising comprising obtaining a CPZEN-45 salt and a capreomycin salt in a weight ratio of 30:70 to 70:30, or of 40:60 to 60:40, or of 45:55 to 55:45, or of 48:52 to 52:48, or of 50:50, preparing a feed solution comprising the CPZEN-45 salt and the capreomycin salt at the above weight ratio in water or buffered aqueous solution, wherein the feed solution optionally does not comprise an excipient or does not comprise an amino acid excipient and/or a surfactant excipient or optionally consists essentially of CPZEN-45 and capreomycin salts and the water or buffered aqueous solution, subjecting the feed solution to spray drying, and collecting resulting spray-dried particles. Thus, in such compositions, the CPZEN-45 and capreomycin are mixed without addition of excipient prior to forming the spray-dried powder. In some embodiments, the spray-dried powder is formed from a mixture consisting essentially of CPZEN-45 and capreomycin in water.

This process contrasts with prior processes of preparing capreomycin spray-dried compositions, for example, in which a significant weight percentage of excipient such as leucine (e.g. at least 5 wt %, such as 10 wt % or 20 wt %) may be added to the capreomycin before spray-drying, presumably to help stabilize the capreomycin. (See WO2007/011396.) When capreomycin was mixed with CPZEN-45, no such stabilizing excipient was necessary to provide a composition with long-term stability, such as up to 6 months in accelerated stability test studies.

The compositions herein may be intended for administration to a subject in need thereof, in which case the composition is a pharmaceutical composition, for example, comprising a unit dosage form of the API or APIs in the composition. Optionally, the dose of CPZEN, e.g., CPZEN-45, in the unit dosage form is 0.1-10 g, or 0.5-5 g, or 1-10 g, or 1-5 g. In some embodiments, the composition is intended for non-oral administration, such as parenteral, subcutaneous, injection, topical, inhalation, intranasal administration, or administration in aerosol form such as with a nebulizer or inhaler device.

The composition may optionally be stored in a container, for example a container that excludes moisture. In this manner, the composition stays dry and in the form of a free-flowing powder. Such a container may include, for example, a vial such as a single-use vial containing one unit dosage form. In some embodiments, the composition present in the container is intended for non-oral administration, such as parenteral, subcutaneous, injection, topical, inhalation, intranasal administration, or administration in aerosol form such as with a nebulizer or inhaler device.

### Administration of Compositions

The present disclosure is based on the discovery that a combination of a CPZEN and a second API, such as capreomycin, provides a treatment for drug resistant forms of tuberculosis, when this combination is delivered by non-oral routes. An exemplary non-oral route is inhalation. To achieve inhalation, any of a nebulizer, a spray bottle or a nasal inhaler may be used to administer a composition of the present disclosure. Other suitable non-oral routes include injection. The composition should be formulated in a manner suitable for the selected route and manner of administration. For example, the composition may contain only pharmaceutically active ingredients and water. Alternatively, the composition may be formulation as a liposome.

In one embodiment the non-oral route includes nebulization. Administration via nebulization typically utilizes a nebulizer. A nebulizer is a machine that turns liquid into a mist so that it can be inhaled with ease and delivered deeply into the lung tissue. The liquid that would be nebulized according to the present disclosure would include water, CPZEN-45, and at least one other antibiotic as described herein. There are many kinds of nebulizer devices available in the marketplace. For example, an ultrasonic nebulizer nebulises liquids very quickly and very quietly using ultrasonic waves to achieve conversion of liquid medicine into a fine mist by a process known as ultrasound atomization. A portable ultrasonic nebulizer is effective, versatile; and can be used at home and during travel; for both adults and children with some modification of delivery tubing. These devices deliver very fine vapors/mist for effective absorption of therapeutic agents deep in the respiratory system-bronchioles and alveoli.

A suitable alternative is the use of a jet nebulizer, which uses air or oxygen under high pressure to generate the aerosol (see, e.g., Luyt CE, Combes A, Nieszkowska A, Trouillet J L, Chastre J. Aerosolized antibiotics to treat ventilator-associated pneumonia. Curr Opin Infect Dns. 2009;22:154-158). During mechanical ventilation, nebulizers are connected to the inspiratory limb of the ventilator circuit, and the composition of the present disclosure can be administered continuously or only during inspiration. See, e.g., Quon, B. S, Ann Am Thorac Soc. 2014 March; 11(3): 425-434 for additional discussion of inhalation methods that can be used with the composition of the present disclosure.

A discussion of the nebulization and administration of pharmaceutically active ingredients and combinations thereof may be found, e.g., in “Nebulizer therapy. Guidelines. British Thoracic Society Nebulizer Project Group” as published in Thorax 1997;52;4-24. See also Eur. Respi. J. 2001; 18: 228-242.

For example, to deliver a composition of the present disclosure through a nebulizer, the patient is asked to stay in a recumbent position on a recliner or bed. The patient is fitted with a facemask or a mouthpiece. If a mask is used, it should be placed comfortably and securely on the face around the mouth of the patient. If a mouthpiece is used, it should be placed between the teeth and the lips of the patient should seal around the tube. The patient takes slow, deep breaths through the mouth, while holding each breath for five to fifteen seconds before breathing out. This allows droplets of the therapeutic agents to settle into the airways and alveoli. Breathing from the diaphragm allows the mist with therapeutic agents to fill and empty the lungs with the mist.

### Bacterial Infections and Diseases to be Treated

Compositions of this disclosure may be useful in treating bacterial infections, for example Mycobacteria infections. In some embodiments, compositions of this disclosure may be useful in treating non-tuberculosis Mycobacteria infections (NTMs). NTMs may, in some embodiments, involve infection of the lungs. NTMs include, for example, infections from species such as M. abscessus, M. abscessus massiliense, M. chelonae, M. kansasii, M. xenopii, M. intracellulare, M. fortuitum, M. ulcerans, M. smegmatis, M. marinum, M. peregrinum, M. mucogenicum, M. alvei, M. porcinum, M. septicum, M. wolinskyi, M. lentiflavum, M. mageritense, M. phlei, M. vaccae, M. malmoense, M. gordonae, M. simiae, M. scrofulaceum, M. hibermiae, M. bovis, and M. avium, which may infect humans or other animals. In other embodiments, compositions of this disclosure may be useful in treating tuberculosis (TB), such as multidrug resistant (MDR) or extensively drug resistant (XDR) tuberculosis. In some embodiments, the TB symptoms are associated with infection from M. tuberculosis. For example, both CPZEN-45 and capreomycin have individually shown utility against NTM and TB infection in model systems. For example, Y. Takahashi et al., J. Antibiotics, 66: 171-178 (2013), provides data on activities of CPZENs including CPZEN-45, 48, and 51, against various Mycobacteria strains including TB and NTM strains. See also I. Soni et al., J. Med. Microbiol. 65: 1-8 (2016), and U.S. Pat. Nos. 8,058,247 and 9,040,502. P. Le Conte et al., Antimicrobial Agents and Chemotherapy 38(12): 2695-2701 (1994), presents data on the activity of liposomal capreomycin against M. avium complex (MAC) infections.

In one aspect, the present disclosure is directed to inhaled TB therapy for the treatment of multiple drug resistant and extensively drug resistant disease. A variety of drugs have been evaluated for pulmonary delivery as dry powders. Notably, capreomycin sulfate has shown efficacy in the guinea pig infection model and was subsequently delivered in large, up to 300 mg, doses to healthy human volunteers with no ill effects. CPZEN-45, also known as Caprazene-45, is a derivative of caprazamycin that has been shown to be effective in treating disease in the guinea pig infection model. In one embodiment, the present disclosure combines these drugs, which fundamentally act by different mechanisms, into a single composition, e.g., a composition preparing by spray drying, to afford an intimate mixture for combination drug therapy. The spray dried combination powder may be prepared in an aerodynamic particle size range (1-5 μm) suitable for pulmonary delivery when delivered from a commercial inhaler, e.g., the Cyclohaler™ device. Chemical and physical storage stability was demonstrated for a period of 6 months. It can be concluded that a combination inhaled drug product containing capreomycin sulfate and CPZEN-45 hydrochloride can be prepared in a stable form suitable to pulmonary delivery to treat tuberculosis.

The rise of MDR and XDR TB as well as the absence of new orally available drugs requires new therapeutic strategies. The present disclosure looks at new forms of delivery for combinations of anti-TB drugs. Although there is currently no FDA-approved inhaled treatment for tuberculosis, the present disclosure recognizes this mode of administration has several advantages. Pulmonary drug delivery can be used to provide much higher acute drug concentration localized to the lungs, thereby expediting bacterial clearance and preventing transmission. Particulate, vesicular, or macromolecular drugs introduced to the lung can be phagocytosed by alveolar macrophages that are targets of M. tuberculosis colonization. The high local concentrations afforded by inhaled therapy can reduce the systemic burden of more potent, potentially toxic second-line drugs like capreomycin. Additionally, drugs given by this route can enter systemic circulation via absorption from the lungs avoiding hepatic first-pass and destruction through liver metabolism thereby increasing bioavailability.

### WORKING EXAMPLES

### Example 1: Preparation of a CPZEN-45 and Capreomycin Spray-Dried Composition

### 1. Preparation of the Composition

CPZEN-45 hydrochloride was received from the Infectious Disease Research Institute (IDRI, Seattle WA) under the umbrella of the TB Lilly Drug Discovery Initiative. Capreomycin sulfate (CS) was purchased from MP Biomedical. CPZEN-45 and related CPZEN compounds may also be prepared by synthesis, see, e.g., Nakamura H. Org. Lett., 2016, 18 (9), pp 2300-2303, Takeuchi, T. Tet. Lett. Volume 57, Issue 26, 29 Jun. 2016, Pages 2901-2904, PCT Publication Nos. WO 2009/094563, WO 2004/067544, WO 2001/12643 and U.S. Pat. No. 8,299,291. Particles were prepared by spray drying (B-290, Buchi, Flawil, Switzerland). A feed solution was prepared by combining equal parts CPZEN-45 and CS in ultrapure water (18.2 MΩ·cm) to a total solids concentration of 10 mg/mL. This feed solution was atomized via two fluid nozzle (nozzle diameters) using nitrogen as the atomizing gas at a liquid flow rate of 6-7 mL/min and a gas flow rate of 439 L/hour into the drying chamber. Room air was used as the drying gas, aspirated through the instrument at a rate of 35 m³/h with a set inlet temperature of 190° C. The particles were collected from the airstream by cyclone using the instrument's standard cyclone and collection vessel.

### 2. Particle Surface Features and Aerodynamics

Particle surface features were visualized by scanning electron microscopy (FEI Quanta) at an accelerating voltage of 15 kV and a spot size of 3.0 under high vacuum at various magnifications. Samples were prepared for imaging by depositing powder onto a carbon adhesive substrate mounted to an aluminum sample holder and sputter coated with Au/Pt (instrument) under argon for a duration of 120 s.

Aerodynamic performance of the aerosol was evaluated at intervals by inertial impaction (NGI, MSP Corp., MN, USA). Powder was loaded into #3 hydroxypropylmethylcellulose (HPMC) capsules (Quali-V®, Qualicaps, N.C., USA) with each capsule containing 10 mg. Capsules were administered to the impactor via a capsule based dry powder inhaler system (Cyclohaler™, Plastiape, Italy). Three capsules were delivered to the impactor sequentially per sample. The impactor was operated at a flow rate of 60 liters per minute for a time of 4 seconds per actuation. Stages were coated by applying 1% w/v silicone oil in hexane and evaporating to minimize particle bounce, and a pre-separator was used to remove oversized aggregates. Drug mass was recovered from each stage with deionized water and analyzed by HPLC. Cumulative drug mass for each API was converted to a log-probability scale and plotted against the effective cutoff diameter of the respective stages to determine the mass median aerodynamic diameter (MMAD) and the geometric standard deviation (GSD). Delivered dose was determined using a dosage unit sampling apparatus (Copley Scientific, Nottingham, UK) in accordance with USPsection <601> operated at a flow rate of 60 SLPM. For each evaluation, 3 capsules were loaded and administered sequentially. Deposition was quantified in the capsules, inhaler and dosing apparatus by HPLC. Delivered dose was expressed as a percentage of the loaded dose.

### 3. Evaluation of Long-Term Stability by Forced Degradation and Accelerated Stability Assays

Prior to evaluating the long-term stability of a spray-dried combination powder, multiple forced degradations on capreomycin and CPZEN-45 were performed before determining the chromatographic profile of treated materials and monitoring absorbance at 265 nm using a Shimadzu RP-HPLC unit. By this method, it was confirmed that it was possible to detect drug degradation as well as any degradants generated.

Forced degradations were performed by subjecting aqueous solutions of non-spray-dried capreomycin or CPZEN-45 at 0.1 mg/mL to multiple conditions (all at 23° C. unless noted otherwise): control (no treatment, 72 hours), acid (0.1 M HCl, pH=1.2, 72 hours), base (0.1 M NaOH, pH=12.8, 72 hours), oxidizer (3% w/v 2Na₂CO₃ 3H₂O₂ (sodium percarbonate), 72 hours), 60° C. for 72 hours, or 90° C. for 30 minutes.

Accelerated stability studies were conducted by storing drug combinations at 25° C./60% relative humidity (RH), 30° C./75% RH and 40° C./75% RH. Drugs were stored either individually as a free-flowing powder, as a 1:1 spray-dried formulation of capreomycin and CPZEN-45 as a free-flowing powder, or as a 1:1 spray-dried formulation in #3 hydroxypropylmethylcellulose (HPMC) capsules. All samples were deposited into 5 mL screw-top glass vials. Samples were collected at the beginning of the study, after 1 week, 1 month, 3 months, and 6 months. HPLC-based methods for detecting CPZEN-45, capreomycin, and degradation products are described in the “Methods” section below.

It was determined that capreomycin is highly sensitive to acid, base, and oxidizer treatment as well as moderately sensitive to high heat. In contrast, CPZEN-45 is quite robust and only sensitive to base and oxidizer treatment. Base treatment results in a loss of approximately 88% of the original mass of intact CPZEN-45.

Specifically, results of accelerated stability studies are as shown in the tables below.

TABLE 1
25° C./60% RH CPZEN-45 API: Results for Assay and Related Substances by HPLC

| TESTING | | | T = 1 | T = 1 | T = 3 | T = 6 |
|---|---|---|---|---|---|---|
| FOR | TIME | T = 0 | WEEK | MON | MON | MON |
| CPZEN-45 | Replicate 1 | 98.1% | 96.2% | 100.7% | N/A | 97.2% |
| | Replicate 2 | 98.0% | 95.8% | 98.0% | 98.0% | 98.4% |
| | Replicate 3 | 95.3% | 97.1% | 99.5% | 103.2% | 97.1% |
| | Average ± RSD | 97.1% ± 1.6% | 96.4% ± 0.7% | 99.4% ± 1.4% | 100.6% ± 3.5% | 97.7% ± 0.9% |
| Related | Replicate 1 | 1.1% | 0.5% | 0.6% | 0.4% | 0.8% |
| Substances | Replicate 2 | 1.1% | 0.4% | 0.7% | 0.6% | 0.9% |
| | Replicate 3 | 1.1% | 0.5% | 0.6% | 0.5% | 0.9% |
| | Average | 1.1% | 0.5% | 0.6% | 0.5% | 0.9% |

TABLE 2
40° C./75% RH CPZEN-45 API: Results for Assay and Related Substances by HPLC

| TESTING | | | T = 1 | T = 1 | T = 3 | T = 6 |
|---|---|---|---|---|---|---|
| FOR | TIME | T = 0 | WEEK | MON | MON¹ | MON |
| CPZEN-45 | Replicate 1 | 98.1% | 96.1% | 100.3% | 97.2% | 96.5% |
| | Replicate 2 | 98.0% | 96.4% | 96.0% | 101.0% | 98.0% |
| | Replicate 3 | 95.3% | 95.9% | 94.4% | 102.8% | 97.5% |
| | Average ± RSD | 97.1% ± 1.6% | 96.1% ± 0.3% | 96.9% ± 3.2% | 100.3% ± 2.9% | 97.3% ± 0.8% |
| Related | Replicate 1 | 1.1% | 0.4% | 2.5% | 0.6% | 1.4% |
| Substances | Replicate 2 | 1.1% | 0.4% | 1.8% | 0.6% | 1.4% |
| | Replicate 3 | 1.1% | 0.5% | 2.0% | 0.8% | 1.2% |
| | Average | 1.1% | 0.4% | 2.1% | 0.7% | 1.3% |

¹Due to the melting of substances at 40° C./75% RH values after month 3 are 30° C./60% RH.

TABLE 3
25° C./60% RH Capreo API: Results for Assay and Related Substances by HPLC

| TESTING | | | T= 1 | T = 1 | T = 3 | T = 6 |
|---|---|---|---|---|---|---|
| FOR | TIME | T = 0 | WEEK | MON | MON | MON |
| Capreo | Replicate 1 | 98.4% | 94.9% | 100.3% | 100.9% | 80.3% |
| | Replicate 2 | 94.9% | 94.1% | 101.1% | 101.8% | 86.1% |
| | Replicate 3 | 96.9% | 88.9% | 100.5% | 101.9% | 88.2% |
| | Average ± RSD | 96.7% ± 1.8% | 92.6% ± 3.5% | 100.6% ± 0.4% | 101.5% ± 0.5% | 84.9% ± 4.8% |
| Related | Replicate 1 | 1.2% | 0.6% | 0.8% | 0.8% | 0.8% |
| Substances | Replicate 2 | 1.4% | 0.5% | 0.9% | 0.9% | 1.0% |
| | Replicate 3 | 1.4% | 0.6% | 0.8% | 0.7% | 1.0% |
| | Average | 1.3% | 0.6% | 0.8% | 0.8% | 0.9% |

TABLE 4
40° C./75% RH Capreo API: Results for Assay and Related Substances by HPLC

| TESTING | | | T = 1 | T = 1 | T = 3 | T = 6 |
|---|---|---|---|---|---|---|
| FOR | TIME | T = 0 | WEEK | MON | MON¹ | MON |
| Capreo | Replicate 1 | 98.4% | 90.1% | 93.8% | 100.0% | 82.0% |
| | Replicate 2 | 94.9% | 88.5% | 92.7% | 105.2% | 86.7% |
| | Replicate 3 | 96.9% | 90.3% | 92.3% | 100.9% | 87.3% |
| | Average ± RSD | 96.7% ± 1.8% | 89.6% ± 1.1% | 92.9% ± 0.9% | 102.0% ± 2.7% | 85.3% ± 3.4% |
| Related | Replicate 1 | 1.2% | 0.5% | 1.0% | 0.9% | 1.1% |
| Substances | Replicate 2 | 1.4% | 0.5% | 1.0% | 0.8% | 1.1% |
| | Replicate 3 | 1.4% | 0.6% | 1.0% | 0.8% | 1.1% |
| | Average | 1.3% | 0.5% | 1.0% | 0.8% | 1.1% |

¹Due to the melting of substances at 40° C./75% RH values after 3 months are 30° C./60% RH.

TABLE 5
25° C./60% RH CPZEN-45/Capreo combo: Results for Assay and Related Substances by HPLC

| TESTING | | | T = 1 | T = 1 | T = 3 | T = 6 |
|---|---|---|---|---|---|---|
| FOR | TIME | T = 0 | WEEK | MON | MON | MON |
| Capreo | Replicate 1 | 46.8% | 45.9% | 52.1% | 54.5% | 46.0% |
| | Replicate 2 | 47.7% | 45.9% | 52.0% | 54.5% | 44.9% |
| | Replicate 3 | 47.8% | 42.6% | 52.1% | 53.0% | 44.6% |
| | Average | 47.4% ± 1.1% | 44.8% ± 4.3% | 52.0% ± 0.1% | 54% ± 1.7% | 45.2% ± 0.6% |
| CPZEN | Replicate 1 | 44.3% | 44.6% | 44.5% | 45.2% | 43.0% |
| | Replicate 2 | 45.0% | 44.6% | 44.4% | 45.0% | 42.5% |
| | Replicate 3 | 45.1% | 41.4% | 44.6% | 43.8% | 41.9% |
| | Average | 44.8% ± 1.0% | 43.5% ± 4.3% | 44.5% ± 0.2% | 44.7% ± 1.7% | 42.5% ± 1.3% |
| Total | Replicate 1 | 91.1% | 90.5% | 96.6% | 99.7% | 89.0% |
| | Replicate 2 | 92.7% | 90.6% | 96.3% | 99.5% | 87.4% |
| | Replicate 3 | 92.9% | 84.0% | 96.6% | 96.8% | 88.3% |
| | Average | 92.2% ± 1.0% | 88.4% ± 3.8% | 96.5% ± 0.2% | 98.7% ± 1.6% | 87.6% ± 1.2% |
| Related | Replicate 1 | 2.0% | 1.6% | 2.0% | 2.3% | 4.0% |
| Substances | Replicate 2 | 1.9% | 1.7% | 2.0% | 2.1% | 3.8% |
| | Replicate 3 | 1.8% | 1.7% | 2.1% | 2.4% | 3.7% |
| | Average | 1.9% | 1.7% | 2.0% | 2.3% | 3.8% |

TABLE 6
40° C./75% RH CPZEN-45/Capreo combo: Results for Assay and Related Substances by HPLC

| TESTING | | | T = 1 | T = 1 | T = 3 | T = 6 |
|---|---|---|---|---|---|---|
| FOR | TIME | T = 0 | WEEK | MON | MON¹ | MON |
| Capreo | Replicate 1 | 46.8% | 46.2% | 49.8% | 52.5% | 45.1% |
| | Replicate 2 | 47.7% | 45.4% | 50.1% | 52.1% | 45.2% |
| | Replicate 3 | 47.8% | 44.9% | 50.0% | 52.1% | 45.7% |
| | Average ± RSD | 47.4% ± 1.1% | 45.5% ± 1.4% | 50.0% ± 0.4% | 52.2% ± 0.4% | 45.3% ± 0.6% |
| CPZEN | Replicate 1 | 44.3% | 44.7% | 42.5% | 43.6% | 42.2% |
| | Replicate 2 | 45.0% | 44.2% | 42.6% | 43.2% | 42.4% |
| | Replicate 3 | 45.1% | 44.0% | 42.6% | 43.3% | 42.7% |
| | Average ± RSD | 44.8% ± 1.0% | 44.3% ± 0.8% | 42.5% ± 0.2% | 43.4% ± 0.5% | 42.4% ± 0.6% |
| Total | Replicate 1 | 91.1% | 90.9% | 92.2% | 96.1% | 87.3% |
| | Replicate 2 | 92.7% | 89.6% | 92.8% | 95.3% | 87.6% |
| | Replicate 3 | 92.9% | 88.9% | 92.6% | 95.4% | 88.3% |
| | Average ± RSD | 92.2% ± 1.0% | 89.8% ± 1.0% | 92.5% ± 0.3% | 95.6% ± 0.4% | 87.7% ± 0.5% |
| Related | Replicate 1 | 2.0% | 2.1% | 2.9% | 2.5% | 4.2% |
| Substances | Replicate 2 | 1.9% | 2.0% | 2.8% | 2.5% | 4.1% |
| | Replicate 3 | 1.8% | 2.0% | 2.8% | 2.5% | 4.2% |
| | Average | 1.9% | 2.0% | 2.8% | 2.5% | 4.2% |

¹Due to the melting of substances at 40° C./75% RH values after 3 months are 30° C./60% RH.

The accelerated stability studies showed capreomycin, CPZEN-45, and the 50:50 combination thereof to be highly stable at both 25° C./60% RH and 30° C./60% RH. Even after 6 months at 30° C. and 60% RH, degradants only made up about 4% of the total mass of the spray-dried drug combination.

### 4. Thermogravimetric Analysis

Thermogravimetric analysis was performed (TGA, TA Instruments, New Castle, Delaware, USA) at stability time points on bulk spray dried powder to determine residual moisture content. Briefly, a platinum sample pan was tarred and approximately 10 mg of powder was loaded and heated under nitrogen purge at a ramp of 5° C. per minute from 20° C. to 300° C. Residual moisture content was determined as the percentage weight lost at the minimum rate of change after 100° C. before decomposition, approximately occurring between 100° C. and 150° C.

### 5. Mucin Binding

Mucin binding was evaluated using a dialysis assay developed previously (Huang et al., 2015). Briefly, 100 μL of capreomycin, CPZEN-45, or a 50:50 combination of both at 4 mg/mL per drug was suspended in 900 μL Dulbecco's PBS without calcium or magnesium (Lonza, Cat. #17-512F) either with or without 12.5% (wt./vol) porcine stomach mucin (Sigma-Aldrich, Cat. #M1778, type III, bound sialic acid 0.5% to 1.5%, partially purified powder). The resulting mixtures were placed in Spectra/Por dialysis bags (6- to 8-kDa molecular mass cutoff, 32-mm flat width, 20.4-mm diameter; 3.3-ml/cm volume; Spectrum Laboratories, Cat. #132655, Rancho Dominguez, Calif.), with the tube ends closed using standard dialysis bag closures. Dialysis was conducted in 10 ml of DPBS at 37° C. in a 9.4-cm polystyrene petri dish (Greiner Bio-One, Cat. #633181) covered with a lid and agitated on an incubated orbital shaker at 100 rpm. Dialysate samples (200 μl) were collected at 10, 60, 120, and 240 minutes and the antibiotic concentration within each dialysate was determined by HPLC UV/vis analysis. The percentage of each antibiotic detected in the dialysate was calculated relative to the theoretical total concentration based on 400 μg of antibiotic in an 11-ml total volume. The study was repeated with the antibiotics colistin sulfate and ciprofloxacin.

Although both capreomycin and CPZEN-45 appear to bind mucin to an extent, the dialysis profiles obtained are similar to the drug ciprofloxacin, which unlike colistin sulfate has been found to suffer only moderate inhibition in the presence of mucin (Huang et al., 2015). After 4 hours the percentages of efflux in the presence of mucin for capreomycin, CPZEN-45, capreomycin in combination with CPZEN-45, and CPZEN-45 in combination with capreomycin were 44%, 48%, 73%, and 69%, respectively, relative to samples without mucin. For comparison, the 4-hour percentage of efflux relative to samples without mucin for colistin sulfate and ciprofloxacin were 20% and 82%. Since any drug that is to be inhaled will have be absorbed through the lungs without being bound and inhibited by pulmonary mucus, these results indicate that an inhaled combination of capreomycin and CPZEN-45 will be unbound and available to treat tuberculosis infection.

### 6. Methods

Capreomycin and CPZEN-45 content for forced degradation, stability, and impactor studies was measured by RP-HPLC with a Shimadzu system consisting of an SCL-10AVP system controller, two LC-10ADVP pumps, a DGU-14A degasser, SIL-10A autoinjector, and SPD-M10AVP diode array detector. The column was an Agilent Zorbax Eclipse XDB-C18, 3.5 μm particle size, 4.6×150 mm². All peaks were detected at wavelength 265 nm. Linearity for total capreomycin was obtained between 1 and 75 μg/mL (R²=0.996) with a limit of detection (LOD) of 0.74 μg/mL. Linearity for CPZEN-45 was obtained between 0.5 and 100 μg/mL (R²=0.996) with an LOD of 1.03 μg/mL.

For forced degradations the mobile phase consisted of 0.1% (v/v) trifluoroacetic acid (TFA) in HPLC-grade H₂O (solvent A) and 0.1% TFA in HPLC-grade acetonitrile (solvent B), delivered at 1 mL/min under the following gradient: 0-6 min, 5% B; 6-8.5 min, 5%-40% B; 8.5-11.5 min, 40% B; 11.5-11.6 min, 40%-95% B; 11.6-13 min, 95% B; 13-13.1 min, 95%-5% B; 13.1-16 min, 5% B. Capreomycin sulfate powder is composed of four distinct compounds that elute at two distinct retention times: capreomycin IA/IB and IIA/IB. Capreomycin IA and IB combine for no less than 90% of total mass. The capreomycin IIA/IIB peak eluted at approximately 2.8 minutes, capreomycin IA/IB at 3.2 minutes, and CPZEN-45 at 11.7 minutes.

For stability and impactor studies, the mobile phase consisted of 0.1% heptafluorobutyric acid (HFBA) in HPLC-grade H₂O (solvent A) and 0.1% TFA in HPLC-grade acetonitrile (solvent B), delivered at 1 mL/min under the following gradient: 0-1.5 min, 20% B; 1.5-10 min, 20%-50% B; 10-11 min, 50% B; 11-11.1 min, 50%-20% B; 11.1-14 min, 20% B. The capreomycin IIA/IIB eluted at approximately 5.8 minutes, capreomycin IA/IB at 6.9 minutes, and CPZEN-45 at 10.1 minutes.

Serum drug concentration was measured via serum spike recovery on an Agilent 6460 Triple Quadrupole LC/MS System with an Acquity UPLC HSS T3 1.8 μM 2.1×100 mm MVK column. The mobile phase consisted of 0.1% HFBA in HPLC-grade H₂O (solvent A) and 0.1% formic acid (FA) in HPLC-grade acetonitrile (solvent B), delivered at 0.3 mL/min under the same gradient as that used for the stability and impactor studies. Samples were prepared by spiking 10 μL 10× stock solutions of spray-dried capreomycin:CPZEN-45 powder into 90 μL guinea pig or human serum, followed by precipitation of serum proteins with 2 μL 70% (v/v) perchloric acid and centrifugation at 16,000 g for 10 minutes. If after one vortex and spin the supernatant was insufficiently clear, another round of vortexing and centrifugation was performed. HPLC-MS/MS was performed on the final supernatant and peak areas associated with compound m/Z's outlined in Table were measured to generate concentration curves.

Ciprofloxacin content was measured by RP-HPLC at 265 nm with an Agilent 1200 series system. The column was an Agilent Zorbax Eclipse XDB-C18, 3.5 μm particle size, 4.6×150 mm². The mobile phase consisted of 0.1% heptafluorobutyric acid (HFBA) in HPLC-grade H₂O (solvent A) and 0.1% TFA in HPLC-grade acetonitrile (solvent B), delivered at 1 mL/min under the following gradient: 0-1.5 min, 20% B; 1.5-10 min, 20%-50% B; 10-11 min, 50% B; 11-11.1 min, 50%-20% B; 11.1-14 min, 20% B. The ciprofloxacin peak eluted at approximately 6.2 minutes.

Colistin sulfate content was measured via mass spectrometry on an Agilent 6460 Triple Quadrupole LC/MS System with a Waters Cortecs UPLC C18 1.6 2.1×50 mm column. The mobile phase consisted of 0.1% formic acid (FA) in HPLC-grade H₂O (solvent A) and 0.1% FA in HPLC-grade acetonitrile (solvent B), delivered at 0.3 mL/min under the following gradient: 0-1 min, 2% B; 1-4 min, 2%-100% B; 4-5 min, 100% B; 5-5.1 min, 100%-2% B; 5.1-7 min, 2% B. The colistin sulfate peak eluted at approximately 3.0 minutes at the m/Z listed in Table 7.

Aerosol performance at 25° C. and 60% RH was consistent over six months, with an average diameter of 2.80 μm (RSD=2.8%)

TABLE 7
Compound m/Z's and Retention Times

| | Parent m/Z → | Approximate Retention |
|---|---|---|
| Compound | Daughter m/Z | Time (minutes) |
| Capreomycin IA | 669.4 → 199.2 | 4.9-5.1 |
| Capreomycin IB | 653.4 → 98.3 | 4.9-5.1 |
| Capreomycin IIA | 541.3 → 98.2 | 3.6-4.0 |
| Capreomycin IIB | 525.3 → 98.3 | 3.6-4.0 |
| CPZEN-45 | 689.3 → 139.1 | 8.1-8.2 |
| Colistin sulfate | 578.8 → 44.2 | 3.0-3.1 |

As shown below in Table 8, the four components of capreomycin as well as CPZEN-45 were all readily detected in both guinea pig and human serum at levels below the MIC for each drug. For the four capreomycin compounds, the LOD given is the concentration of total capreomycin that must be present for that specific compound to be detected. While total capreomycin must be 3.58 μg/mL for capreomycin IA to be within the detection limit in guinea pig serum, only 0.68 μg/mL total drug must be present for capreomycin IIA to be within the detection limit. Accordingly, it is possible to reliably measure and characterize the relationship between drug dosing and systemic serum concentration in subject enrolled in clinical trials.

TABLE 8
R2 values and limits of detection (LOD) for capreomycin
and CPZEN-45 compounds in guinea pig or human serum.

| | Guinea Pig | | Human | |
|---|---|---|---|---|
| | R² | LOD (μg/mL) | R² | LOD (μg/mL) |
| Capreomycin IA | 0.937 | 3.58 | 0.986 | 1.67 |
| Capreomycin IB | 0.975 | 2.22 | 0.989 | 1.46 |
| Capreomycin IIA | 0.998 | 0.68 | 0.999 | 0.45 |
| Capreomycin IIB | 0.993 | 1.16 | 0.997 | 0.79 |
| CPZEN-45 | 0.998 | 0.57 | 1.000 | 0.15 |

### Example 2: Testing of a CPZEN-45 and Capreomycin Spray-Dried Composition in Guinea Pigs

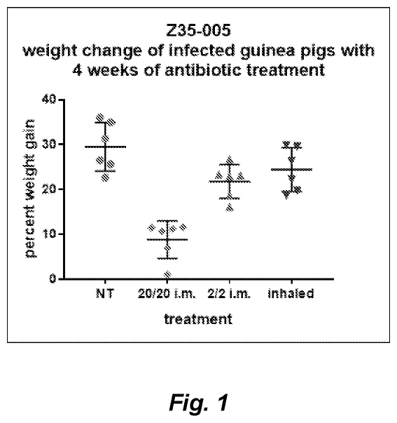

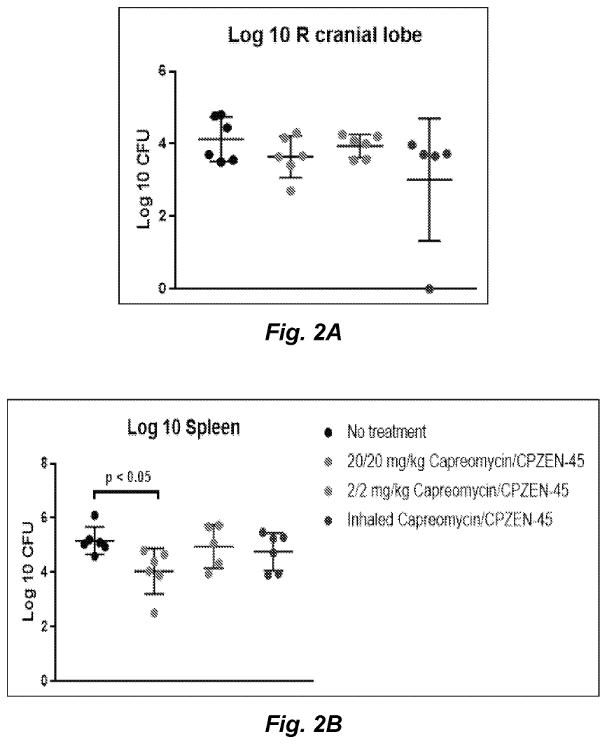

For efficacy testing, 5-10 CFU (colony forming unit) of tuberculosis bacteria were delivered to the lungs of animals using the Mycobacterium tuberculosis strain H37Rv at 2×10⁵ CFU/ml in a nebulizer. Guinea pigs (six per group) were placed into four groups, either an untreated group, or one of three groups for treatment with a spray-dried powder composition of 50:50 CPZEN-45:capreomycin by weight, 20 mg/kg of each drug by intramuscular injection, (20/20 i.m.), 2 mg/kg of each drug by intramuscular injection (2/2 i.m.) or by inhalation (using specially constructed inhalation chamber, animals were dosed for 20 minutes). Animals were dosed 5 days a week for 4 weeks and then sacrificed. To determine efficacy, the right cranial lobe and spleen of the animals were obtained to assess bacterial burden, which is reported in CFU (colony forming unit). Weight change and reduction in CFU in each group are shown in FIGS. 1 and 2A-2B, starting from the untreated group to the left of each graph, followed by the 20/20 i.m., 2/2 i.m., and inhaled treatment groups proceeding from second left to far right in each graph.

Statistical significance between the untreated and treated groups were not reached possibly in part due to the small sizes of each group and the variability in results within each group.

### REFERENCES

2010. WHO global tuberculosis control report 2010. Summary. Central European journal of public health, 18, 237.

BOEHME, C. C., NICOL, M. P., NABETA, P., MICHAEL, J. S., GOTUZZO, E., TAHIRLI, R., GLER, M. T., BLAKEMORE, R., WORODRIA, W., GRAY, C., HUANG, L., CACERES, T., MEHDIYEV, R., RAYMOND, L., WHITELAW, A., SAGADEVAN, K., ALEXANDER, H., ALBERT, H., COBELENS, F., COX, H., ALLAND, D. & PERKINS, M. D. 2011. Feasibility, diagnostic accuracy, and effectiveness of decentralised use of the Xpert MTB/RIF test for diagnosis of tuberculosis and multidrug resistance: a multicentre implementation study. Lancet, 377, 1495-505.

FIEGEL, J., GARCIA-CONTRERAS, L., THOMAS, M., VERBERKMOES, J., ELBERT, K., HICKEY, A. & EDWARDS, D. 2008. Preparation and in vivo evaluation of a dry powder for inhalation of capreomycin. Pharm Res, 25, 805-11.

GELLER, D. E., WEERS, J. & HEUERDING, S. 2011. Development of an inhaled dry-powder formulation of tobramycin using PulmoSphere technology. J Aerosol Med Pulm Drug Deliv, 24, 175-82.

HANIF, S. N., HICKEY, A. J. & GARCIA-CONTRERAS, L. 2014. Liquid chromatographic determination of CPZEN-45, a novel anti-tubercular drug, in biological samples. J Pharm Biomed Anal, 88, 370-6.

HICKEY, A. J. 1996. Inhalation Aerosols: Physical and Biological Basis for Therapy, CRC Press.

HICKEY, A. J. & GANDERTON, D. 2009. Pharmaceutical Process Engineering, Second Edition, Taylor & Francis.

HICKEY, A. J. & SMYTH, H. D. C. 2010. Pharmaco-Complexity: Non-Linear Phenomena and Drug Product Development, Springer US.

HUANG, J. X., BLASKOVICH, M. A., PELINGON, R., RAMU, S., KAVANAGH, A., ELLIOTT, A. G., BUTLER, M. S., MONTGOMERY, A. B. & COOPER, M. A. 2015. Mucin Binding Reduces Colistin Antimicrobial Activity. Antimicrob Agents Chemother, 59, 5925-31.

ISHIZAKI, Y., HAYASHI, C., INOUE, K., IGARASHI, M., TAKAHASHI, Y., PUJARI, V., CRICK, D. C., BRENNAN, P. J. & NOMOTO, A. 2013. Inhibition of the first step in synthesis of the mycobacterial cell wall core, catalyzed by the GlcNAc-1-phosphate transferase WecA, by the novel caprazamycin derivative CPZEN-45. J Biol Chem, 288, 30309-19.

JOHANSEN, S. K., MAUS, C. E., PLIKAYTIS, B. B. & DOUTHWAITE, S. 2006. Capreomycin binds across the ribosomal subunit interface using tlyA-encoded 2′-O-methylations in 16S and 23S rRNAs. Mol Cell, 23, 173-82.

LAWN, S. D., MWABA, P., BATES, M., PIATEK, A., ALEXANDER, H., MARAIS, B. J., CUEVAS, L. E., MCHUGH, T. D., ZIJENAH, L., KAPATA, N., ABUBAKAR, I., MCNERNEY, R., HOELSCHER, M., MEMISH, Z. A., MIGLIORI, G. B., KIM, P., MAEURER, M., SCHITO, M. & ZUMLA, A. 2013. Advances in tuberculosis diagnostics: the Xpert MTB/RIF assay and future prospects for a point-of-care test. Lancet Infect Dis, 13, 349-61.

MISRA, A., HICKEY, A. J., ROSSI, C., BORCHARD, G., TERADA, H., MAKINO, K., FOURIE, P. B. & COLOMBO, P. 2011. Inhaled drug therapy for treatment of tuberculosis. Tuberculosis (Edinb), 91, 71-81.

MUTTIL, P., WANG, C. & HICKEY, A. J. 2009. Inhaled drug delivery for tuberculosis therapy. Pharm Res, 26, 2401-16.

WHO. 2016. Tuberculosis Fact Sheet N° 104 [Online]. Available: http://www.who.int/mediacentre/factsheets/fs104/en/.

1. A spray-dried powder composition comprising CPZEN-45 and capreomycin.

2. The composition of claim 1, wherein CPZEN-45 comprises 10-90 wt %, 20-80 wt %, 30-70 wt %, 40-60 wt %, or 45-55 wt % of total weight of active pharmaceutical ingredient in the composition.

3. The composition of claim 2, wherein CPZEN-45 comprises 40-60 wt%, 45-55 wt %, 45 wt %, 46 wt %, 47 wt %, 48 wt %, 49 wt %, 50 wt %, 51 wt %, 52 wt %, 53 wt %, 54 wt %, or 55 wt % of total weight of active pharmaceutical ingredient in the composition, or wherein the CPZEN-45 and capreomycin are present in a weight ratio range of 40:60 to 60:40, or a weight ratio range of 45:55 to 55:45, or a weight ratio range of 48:52 to 52:48, or a weight ratio of 50:50.

4. The composition of claim 2, wherein CPZEN-45 and capreomycin make up 100% of the total weight of active pharmaceutical ingredient of the composition.

5. The composition of claim 1, wherein the composition consists essentially of CPZEN-45 and capreomycin.

6. The composition of claim 1, wherein CPZEN-45 is in a free base form.

7. The composition of claim 1, wherein CPZEN-45 is in a salt form.

8. The composition of claim 7, wherein CPZEN-45 is in a hydrochloride salt form.

9. The composition of claim 1, wherein capreomycin is in a salt form.

10. The composition of claim 9, wherein capreomycin is in a sulfate salt form (capreomycin sulfate).

11. The composition of claim 1, wherein the composition further comprises at least one other antibiotic.

12. The composition of claim 11, wherein the at least one other antibiotic is an aminoglycoside.

13. The composition of claim 12, wherein the aminoglycoside is selected from isoniazid, pyrazinamide, clarithromycin, azithromycin, rifampin, rifabutin, ethambutol, levofloxacin, moxifloxacin, ofloxacin, clofazimine, clarithromycin, cycloserine, para-aminosalicylic acid, terizidone, thionamide, protionamide, gatifloxacin, bedaquiline, delamanid, meropenem, kanamycin, amikacin, tobramycin, dibekacin, gentamicin, sisomicin, netilmicin, neomycins B, C, neomycin E (paromomycin) and streptomycin.

14. The composition of claim 1, which comprises less than 5 wt % excipients, which comprises less than 4 wt % excipients, which comprises less than 3 wt % excipients, which comprises less than 2 wt % excipients, or which comprises less than 1 wt % excipients, based on the total weight of the composition; or which does not comprise excipients.

15. The composition of claim 1, which does not comprise an amino acid excipient, does not comprise a surfactant excipient, or does not comprise either an amino acid or a surfactant excipient.

16. The composition of claim 1, which contains less than 10 wt % water based on the total weight of the composition.

17. The composition of claim 1, wherein the spray-dried powder composition comprises particles with an average particle size between 0.1 and 10 μm, between 0.5 and 10 μm, between 1 and 5 μm, or between 2 and 4 μm.

18. A spray-dried powder composition consisting essentially of CPZEN-45 salt (e.g. HCl salt) and capreomycin salt (e.g. sulfate salt) and less than 1, 2, 3, 4, or 5 wt % water, wherein the CPZEN-45 and capreomycin are present at a weight ratio range of 45:55 to 55:45, or a weight ratio range of 48:52 to 52:48, or a weight ratio range of 50:50, and wherein the powder has a particle size of between 1 and 5 μm.

19. The composition of claim 18, wherein the spray-dried powder composition is at least 95%, at least 96%, or at least 97% stable against degradation of CPZEN-45 and capreomycin for at least 6 months at 25 ° C. and 60% relative humidity.

20. The composition of claim 19, wherein the CPZEN-45 and capreomycin are mixed without addition of excipient prior to forming the spray-dried powder.

21. The composition of claim 20, wherein the spray-dried powder is formed from a mixture consisting essentially of CPZEN-45 and capreomycin in water.

22. A pharmaceutical composition comprising a unit dosage form of the composition of claim 21.

23. The pharmaceutical composition of claim 22, comprising a unit dose of CPZEN-45 of 0.1-10 g, 1-10 g, or 1-5 g, and/or comprising a unit dose of capreomycin of 0.1-10 g, 1-10 g, or 1-5 g.

24. A container comprising the pharmaceutical composition of claim 22.

25. A composition or pharmaceutical composition or container of claim 24 for use in treating a bacterial infection.

26. The composition, pharmaceutical composition or container for use of claim 25, wherein the bacterial infection is a Mycobacteria infection.

27. The composition, pharmaceutical composition or container for use of claim 26, wherein the Mycobacteria infection is a non-tuberculosis Mycobacteria (NTM) infection.

28. The composition, pharmaceutical composition or container for use of claim 27, wherein the NTM is caused by one or more of M. abscessus, M. abscessus massiliense, M. chelonae, M. kansasii, M. xenopii, M. intracellulare, M. fortuitum, M. ulcerans, M. smegmatis, M. marinum, M. peregrinum, M. mucogenicum, M. alvei, M. porcinum, M. septicum, M. wolinskyi, M. lentiflavum, M. mageritense, M. phlei, M. vaccae, M. malmoense, M. gordonae, M. simiae, M. scrofulaceum, M. hibermiae, M. bovis, and M. avium.

29. The composition, pharmaceutical composition or container for use of claim 25, wherein the bacterial infection is tuberculosis, MDR tuberculosis, or XDR tuberculosis.

30. The composition, pharmaceutical composition or container for use of claim 25, wherein treating the bacterial infection comprises administering the composition or pharmaceutical composition to the subject via a route selected from inhalation, nebulization, parenteral administration, topical administration, and injection.

31. The composition, pharmaceutical composition or container for use of claim 25, wherein treating the bacterial infection comprises administering the composition or pharmaceutical composition in a form of an aerosolized droplet.

32. The composition, pharmaceutical composition or container for use of claim 25, wherein treating the bacterial infection comprises administering the composition as a powder via inhalation.

33. A method of treating a bacterial infection comprising administering to a subject in need thereof the composition or pharmaceutical composition according to claim 1.

34. The method of claim 33, wherein the bacterial infection is a Mycobacteria infection.

35. The method of claim 34, wherein the Mycobacteria infection is a non-tuberculosis Mycobacteria (NTM) infection.

36. The method of claim 35, wherein the NTM comprises one or more of M. abscessus, M. abscessus massiliense, M. chelonae, M. kansasii, M. xenopii, M. intracellulare, M. fortuitum, M. ulcerans, M. smegmatis, M. marinum, M. peregrinum, M. mucogenicum, M. alvei, M. porcinum, M. septicum, M. wolinskyi, M. lentiflavum, M. mageritense, M. phlei, M. vaccae, M. malmoense, M. gordonae, M. simiae, M. scrofulaceum, M. hibermiae, M. bovis, and M. avium.

37. The method of claim 33, wherein the bacterial infection is tuberculosis, MDR tuberculosis, or XDR tuberculosis.

38. The method of claim 33, wherein the composition is administered to the subject via a route selected from inhalation, nebulization, parenteral administration, topical administration, and injection.

39. The method of claim 33, wherein the composition is administered to the subject in a form of an aerosolized droplet.

40. The method of claim 33, wherein the composition is administered to the subject as a powder via inhalation.

41. A method of preparing a spray-dried composition of CPZEN-45 and capreomycin comprising obtaining a CPZEN-45 salt and a capreomycin salt in a weight ratio range of 30:70 to 70:30, or of 40:60 to 60:40, or of 45:55 to 55:45, or of 48:52 to 52:48, or a weight ratio of 50:50, preparing a feed solution comprising the CPZEN-45 salt and the capreomycin salt at the above weight ratio in water or buffered aqueous solution, wherein the feed solution optionally does not comprise an excipient or does not comprise an amino acid excipient and/or a surfactant excipient and optionally consists essentially of the CPZEN-45 salt and the capreomycin salt in the water or the buffered aqueous solution, subjecting the feed solution to spray drying, and collecting resulting spray-dried particles.

42. A spray-dried powder composition consisting essentially of CPZEN-45 salt (e.g. HCl salt) and capreomycin salt (e.g. sulfate salt) and less than 1, 2, 3, 4, or 5 wt % water, wherein the CPZEN-45 and capreomycin are present at a weight ratio range of 45:55 to 55:45, or a weight ratio range of 48:52 to 52:48, or a weight ratio range of 50:50, and wherein the powder has a particle size of between 1 and 5 μm which is prepared according to the method of claim 41.
